(19) United States
(12) Patent Application Publication
(10) Pub. No.: US 2003/0109848 A1
(43) Pub. Date: Jun. 12, 2003

(54) Apparatus and method for selective positioning of feeding tubes

(76) Inventor: Brian J. Fleeman, Wichita, KS (US)

Correspondence Address: HOVEY WILLIAMS TIMMONS & COLLINS, 2405 GRAND BLVD., SUITE 400, KANSAS CITY, MO, 64108

(21) Appl. No.: 09/726,841

(22) Filed: Nov. 30, 2000

(51) Int. Cl. A61J 7/00; A61M 31/00

(52) U.S. Cl. 604/516; 604/77

(57) ABSTRACT

An improved patient feeding tube (10) is provided which includes an elongated, tubular body (22) presenting a distal end (14) adapted to be inserted into the patient, and a proximal portion (16) designed to remain outside of the patient. The tube (10) is equipped with a fixture (26) adjacent the proximal end (18) thereof, with the fixture (26) permitting attachment of a CO₂ detecting machine (20) to the tube (10). In use, the machine (20) is actuated during insertion of the feed tube (10). If during such insertion, the distal end (14) enters the trachea (62) of the patient (12), the presence of CO₂ adjacent the end (14) will be immediately detected. The user may then withdraw the end (14) and reinsert until proper placement within the esophagus (60) of the patient (12) is achieved. The use of feed tube (10) thus minimizes the possibility that distal end (14) will be improperly placed within the patient (12).

### BACKGROUND OF THE INVENTION

1. Field of the Invention

The present invention is broadly concerned with an improved patient feeding tube designed to largely eliminate the problem of improper feeding tube placement in a patient. More particularly, the invention is concerned with such a feeding tube, as well as corresponding methods and feed tube components, wherein the feeding tube is designed to be coupled with a CO₂ detecting machine. In this fashion, the presence of CO₂ adjacent the distal end of the tube is detected during tube insertion, thereby alerting the installer that the tube is improperly placed in the patient's trachea. However, when the distal end of the tube is properly placed in the patient's esophagus, the absence of substantial CO₂ detected by the detecting machine confirms proper placement of the feeding tube.

2.Description of the Prior Art

Nasal and oral inserted feeding tubes are used for the short term feeding (30 days or less) of patients requiring nutritional support. In practice, the tube is inserted either into the mouth or nose of the patient through the patient's pharynx and into the esophagus. A major complication of this process is the potential of passing the feeding tube into the trachea, and then deeper into the respiratory tract. This can cause damage to the respiratory tract, and in serious cases, the death of the patient.

Current methods used to confirm proper placement of feeding tubes in the esophagus include fluoroscopy, chest X-rays and specially adapted stethoscopes. Additionally, specially tipped feeding tubes used along with an external locator device have also been proposed in the past. Fluoroscopy and chest X-rays are time consuming, extremely expensive, and expose the patient and medical staff to high doses of radiation. The specially adapted stethoscopes are difficult to use because of the need to differentiate sounds, especially in noisy hospital environments. The specially tipped feeding tubes and corresponding external locator device systems are also very expensive and require extensive knowledge of anatomical landmarks. As these tubes are advanced, the external locator detects and indicates the tube's position. However, tracheal intubation cannot be ruled out until it is determined by the external locator readout that the tube is past the lungs. Accordingly, as this occurs at too late of a time to prevent damage, this is an ineffectual method of monitoring and confirming feeding placement.

There is accordingly a need in the art for a less expensive and radiation-free way of confirming proper placement of a feeding tube within a patient. Desirably, such a method and apparatus could be used by relatively unskilled personnel, and provide continuous feedback, i.e., eliminating the need for developing X-rays or reviewing fluoroscopic scans.

### SUMMARY OF THE INVENTION

The present invention overcomes the problems outlined above, and provides an improved feeding tube, as well as a method, which allows the installer to easily ascertain whether the tube is properly in place within the patient's esophagus.

Broadly speaking, the feeding tube of the invention is in the form of an elongated tube presenting a distal end adapted for insertion into a patient and a proximal portion designed to remain outside the patient. A fixture is operably coupled with the proximal portion of the tube in order to permit attachment of a CO₂ detecting machine to the tube. In this fashion, the presence of CO₂ adjacent the distal end of the tube may be detected during installation, when the tube is inserted into a patient. Preferably, the fixture is in the form of a tubular, bifurcated body presenting a pair of tubular legs. One of the legs is secured to the proximal end of the feed tube, while the other of the legs is in communication with the first leg and the interior of the feeding tube. The other leg is designed to couple with a CO₂ detecting machine. To this end, one or more intermediate coupling members may be employed for connecting the fixture and the CO₂ detecting machine.

The invention also pertains to a method of placing a feeding tube in a patient, wherein the feeding tube is inserted through the patient's nose or mouth and through the patient's pharynx for passage into and through the patient's esophagus for ultimate placement of the distal end of the tube in communication with the patient's small intestine. The improved method of the invention involves detecting the presence of CO₂ adjacent the distal end of the feed tube during installation thereof. If a substantial or threshold amount of CO₂ is detected, this indicates that the tube is improperly placed in or adjacent the patient's trachea. On the other hand, if no substantial CO₂ is detected, the installer knows that the tube is not improperly placed, but rather is proceeding toward or in the patient's esophagus.

While use of a specialized feeding tube having the CO₂ detecting machine connection fitting thereon is preferred, the invention also embraces use of a fitting for retrofit attachment to existing feeding tubes. Such fixtures preferably comprise a bifurcated body presenting first and second tubular legs with the first leg having a connection end adapted for attachment to the proximal end of an existing feeding tube to form a continuation thereof. The second leg of the fixture is in communication with the first leg and is adapted for connection to a CO₂ detecting machine.

### BRIEF DESCRIPTION OF THE DRAWINGS

FIG. 1 is a view illustrating the preferred feeding tube of the present invention, showing during insertion into a patient and coupled with a CO₂ detecting machine;

FIG. 2 is a fragmentary view depicting the interconnection between the feeding tube and a component of the CO₂ detecting machine, as illustrated in FIG. 1;

FIG. 3 is a vertical sectional view depicting in detail the construction of the proximal end of the feeding tube, illustrating the construction of the bifurcated fixture permitting attachment of a CO₂ detecting machine;

FIG. 4 is a fragmentary view illustrating the proximal end of a feeding tube in accordance with the invention, shown with a different connection assembly between the feeding tube and a CO₂ detecting machine;

FIG. 5 is a fragmentary perspective view depicting another type of CO₂ detecting machine and preferred hardware employed for coupling the detecting machine with the proximal end of a feeding tube; and

FIG. 6 is a vertical sectional view illustrating in detail the connection hardware illustrated in FIG. 5.

### DETAILED DESCRIPTION OF THE PREFERRED EMBODIMENT

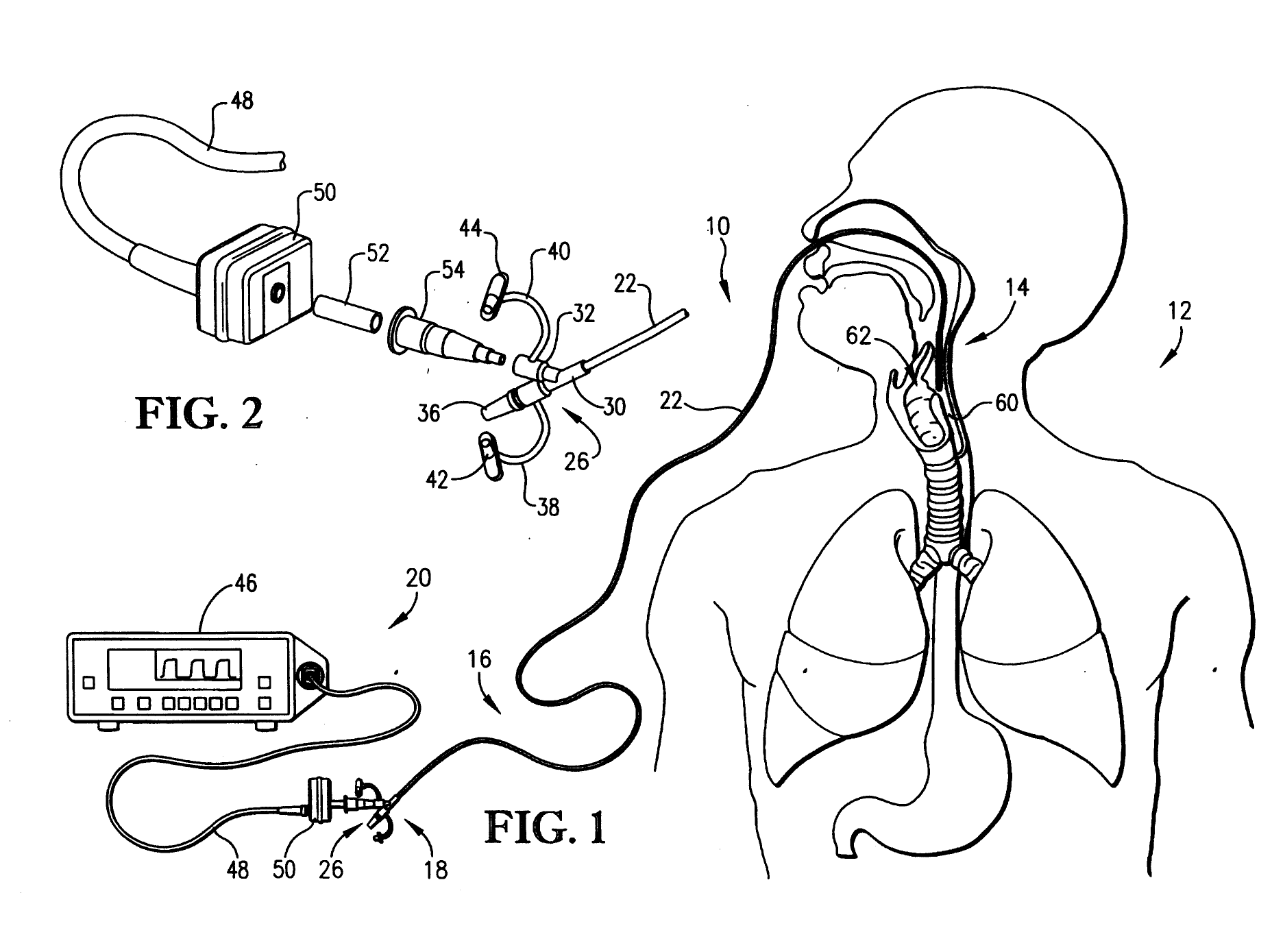

Turning now to FIG. 1, an improved feeding tube 10 in accordance with the invention is shown during insertion thereof into a patient 12. The tube 10 is in the form of an elongated tubular body presenting a distal end 14 and a proximal portion 16 terminating in a proximal end 18. A CO₂ detecting machine 20 is operatively coupled with the proximal portion 16 as will be described below. The tube 10 is designed to permit rapid, easy detection of the proper placement of the tube within patient 12, and to thereby prevent injury or the like resulting from improper placement of the tube in the trachea of the patient.

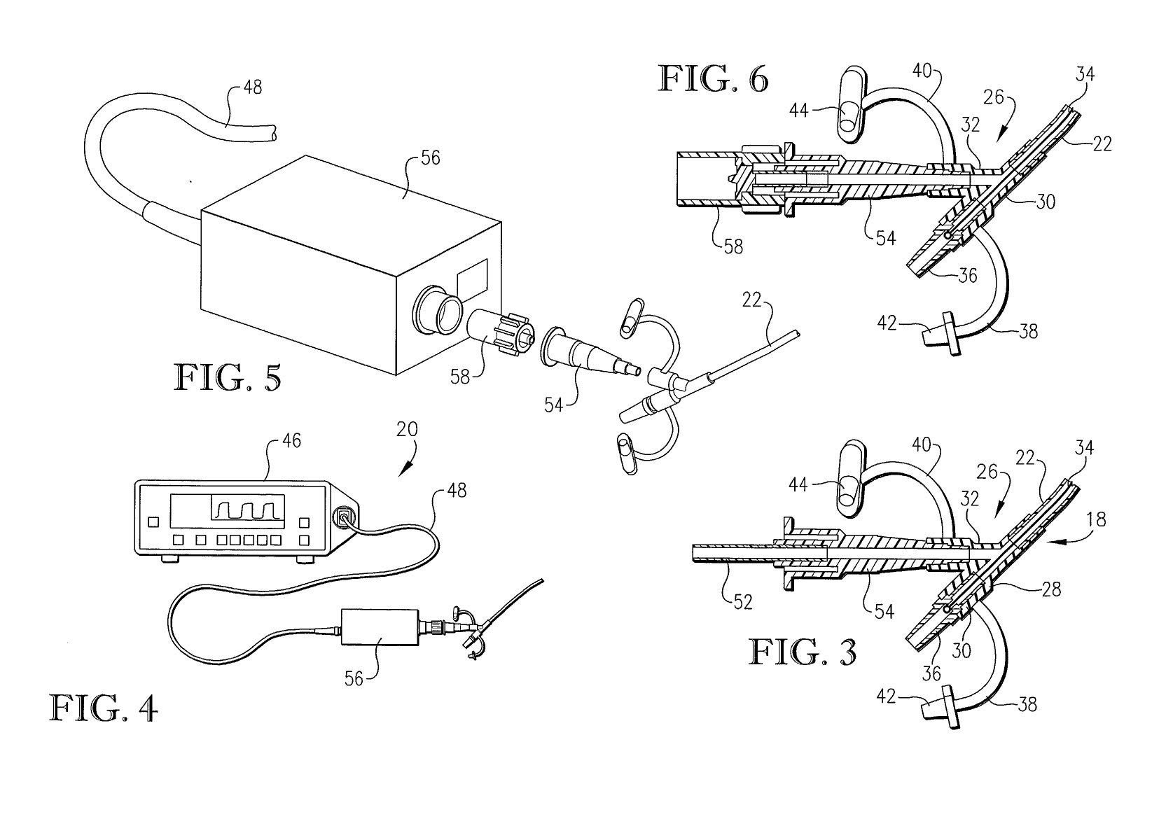

In more detail, the tube 10 is for the most part conventional, and includes an elongated, flexible, synthetic resin tubular body 22 having a conventional proximal end 14 known to those skilled in the art. The tube 10 is modified, however, by provision of an attachment fixture 26 at proximal end 18. Referring to FIG. 3, it will be observed that the fixture 26 is in the form of a somewhat Y-shaped tubular member 28 presenting a first leg 30 and an obliquely oriented leg 32. The leg 30 receives the tip of tubular body 22 as shown, whereby the leg 30 in effect forms a continuation of the tubular body 22. In this regard, it will also be seen that a guide wire 34 extends along the length of tube 22. This guide wire is retained in place by an endmost tubular mount 36 which is secured to the end of leg 30 remote from tube 22. The overall fixture further has a pair of pigtail stoppers 38 and 40 respectively secured to the legs 30, 32 and including a conical stopper projection 42, 44 designed to fit within the ends of the legs 30, 32 as will be described.

The machine 20 in the embodiment of FIG. 1 includes a machine console 46, tubular conduit 48 and connector block 50. In order to properly connect the block 50 with leg 32 of fixture 26, a pair of flexible, tubular, synthetic resin coupling members 52 and 54 are employed. As shown, the member 52 is a simple tubular insert, whereas the member 54 is in the general shape of a cone. The interconnection of the members 50, 52 with fixture 26 is illustrated in FIG. 3; that is, the smaller diameter end of the cone 54 is inserted within the outer end of leg 32, whereas the tube 52 is inserted within the confines of cone 54.

It will be understood that a variety of different CO₂ connectors and connection components can be used in the context of the invention. That is, the invention is not in any way limited to a particular type or style of CO₂ detector, and similarly any suitable connection hardware effecting a proper connection between the machine and the tube 10 can be employed. For example, FIG. 4 illustrates the use of the machine 20 equipped with a different connection block 56, requiring the use of a modified tubular connection member 58 together with cone 54. Again, the interconnection of these components is more specifically illustrated in FIG. 6, where it will be seen that cone 54 is inserted into leg 32, whereas coupler 58 is inserted into the outer, larger diameter end of cone 54. Additionally, cone 54 can be a smoothly tapered cone or any other suitably shaped and sized connector, as opposed to the segmented tapered cone shown in the drawing figures. As would be apparent to one of ordinary skill in the art, the tapering of cone 54 provides adaptability for use with differently sized tubes.

As used herein, the term “CO₂” detector refers to any suitable capnograph or similar device designed to detect the presence and/or amount of carbon dioxide. One device of this character is the NPB-75 handheld capnograph commercialized by the Nellcor Division of Mallinckrodt, Inc. Likewise, another suitable capnograph or capnometer is commercialized by Pulmolink under the designations 8400 or 8200 Capnocheck.

In the use of feeding tube 10, the proximal end 18 thereof is connected with a selected CO₂ detecting machine, such as the machine 20, using the endmost fixture 26. At this point, the user inserts the proximal end 14 of the tube 10 through the patient's nose or mouth as shown in FIG. 1, for passage of the end 14 through the patients pharynx. The goal of course is to insure that the end 14 passes into and through the patient's esophagus 60 rather than into the trachea 62. To this end, during the insertion process, the CO₂ detecting machine is operated so as to detect the presence of CO₂ adjacent the distal end 14 of tube 10. That is, if the end 14 passes into the trachea 62, the presence of CO₂ in substantial quantity within the patient's lungs will be detected by the CO₂ detecting machine, thus signaling to the user that the end 14 is being improperly placed. The tube may then be partially withdrawn and reinserted until the end 14 passes by the trachea opening and into the patients esophagus. In such orientation, little or no carbon dioxide will be detected adjacent end 14, this of course being monitored by the machine 20.

Once the tube 10 has been properly placed with distal end 14 in the patient's esophagus and in communication with the patient's stomach, the tube can be advanced to the small intestine where guide wire 34 is removed. This involves detaching the mount 36 from the proximal end of the tube 10, and pulling the guide wire outwardly in the usual fashion. At this point, the patient may be fed by the usual technique of passing liquid food through the proximal end of the tube for delivery into the patient's small intestine.

While in preferred forms use is made of the specially designed tube 10 having the bifurcated connection fixture adjacent the proximal end thereof, the invention is not so limited. For example, a fixture identical or very similar to the fixture 26 may be provided as a retrofit for existing, conventional feeding tubes. In such an embodiment, the connection end of the fixture leg 30 would be designed to accept the tip end of the conventional feed tube.

I claim:

1. In a method of placing a feeding tube in a patient wherein the feeding tube is inserted through the patient's nose or mouth and through the patient's pharynx for passage into and through the patient's esophagus for ultimate placement of the distal end of the tube in communication with the patient's small intestine, an improved method of determining that said distal tube end is passing into and through the esophagus rather than the patient's trachea, comprising the step of detecting the presence of CO₂ adjacent said distal tube end.

2. The method of claim 1, including the step of detecting the amount of CO₂ adjacent said tube end.

3. The method of claim 2, said amount-detecting step comprising the step of coupling a proximal portion of said tube with a CO₂ detecting machine in order to detect CO₂ passing through the tube from said distal end to said proximal portion.

4. A patient feeding tube comprising:
an elongated tube presenting a distal end adapted for insertion into a patient and a proximal portion designed to remain outside the patient; and
a fixture operably coupled with said proximal portion in order to permit attachment of a CO₂ detecting machine to the tube so that the presence of CO₂ adjacent said distal end may be detected when the tube is inserted into a patient.

5. The feeding tube of claim 4, said tube presenting a proximal end, said fixture comprising a tubular, bifurcated body presenting a pair of tubular legs, one of said legs secured to said proximal end, the other of said legs in communication with the interior of said tube.

6. The feeding tube of claim 5, including one or more intermediate coupling members for connecting said fixture and said machine.

7. The combination comprising:
an elongated patient feeding tube presenting a distal end adapted for insertion into a patient and a proximal portion designed to remain outside the patient; and
a CO₂ detecting machine operably coupled with said proximal portion of said tube so that the presence of CO₂ adjacent said distal end may be detected when the tube is inserted into a patient.

8. The combination of claim 7, including a fixture operably coupled with said proximal portion, said machine coupled with said fixture.

9. The combination of claim 8, said tube presenting a proximal end, said fixture comprising a tubular, bifurcated body presenting a pair of tubular legs, one of said legs secured to said proximal end, the other of said legs in communication with the interior of said tube.

10. The combination of claim 9, including one or more intermediate coupling members for connecting said fixture and said machine.

11. A fixture for connection to the proximal end of a feeding tube, said fixture comprising a bifurcated body presenting first and second tubular legs, said first leg having a connection end adapted for attachment to said proximal end to form a continuation thereof, said second leg in communication with the first leg and adapted for connection with a CO₂ detecting machine.

12. The fixture of claim 11, including a guide wire extending through said first leg and feeding tube, there being a guide wire mount removably secured to the end of said first leg remote from said connection end.
